(19) United States
(12) Patent Application Publication
(10) Pub. No.: US 2025/0169992 A1
(43) Pub. Date: May 29, 2025

(54) TREATMENT OF HEARING LOSS THROUGH AUDITORY TRAINING

(71) Applicant: BELL THERAPEUTICS INC., Seoul (KR)

(72) Inventors: Jonghwa PARK, Seoul (KR); Jeongmi PARK, Suwon-si (KR); Wonwoo LEE, Goyang-si (KR); Jiyeon HA, Yangju-si (KR); Jaeeun LEE, Seoul (KR); Junghoo KIM, Seoul (KR); Joongbae YUM, Seoul (KR)

(21) Appl. No.: 19/037,797

(22) Filed: Jan. 27, 2025

Related U.S. Application Data
(63) Continuation-in-part of application No. PCT/KR2024/095938, filed on Jul. 26, 2024.

(30) Foreign Application Priority Data
Jul. 28, 2023 (KR) 10-2023-0098992
Feb. 19, 2024 (KR) 10-2024-0023586
Jul. 25, 2024 (KR) 10-2024-0098856

(51) Int. Cl. A61F 11/00

(52) U.S. Cl. CPC A61F 11/00

(57) ABSTRACT

The present invention relates to a method for treating hearing loss using auditory feedback. The treatment method according to the present invention provides auditory training sounds to hearing-impaired patients along with visual representations of the sound characteristics and receives input from the patients regarding these sound characteristics. Through this approach, it enables integrated training that combines auditory, visual, and motor feedback for hearing-impaired patients. Compared to traditional voice-centered training methods, this provides a more effective and intuitive learning environment, resulting in superior auditory improvement outcomes. Additionally, since the method can identify sound characteristics to which hearing-impaired patients are vulnerable, it allows for customized treatment tailored to each patient. By intensively training these areas, auditory processing can be improved more efficiently. Furthermore, by including linguistic training as part of multisensory auditory training, it can comprehensively enhance music and language recognition abilities. This leads to effective rehabilitation in various aspects, such as improved speech comprehension, better differentiation of everyday sounds, and enhanced quality of music appreciation.

### CROSS-REFERENCE TO RELATED APPLICATION(S)

This application is a Continuation-In-Part (CIP) application, claiming priority under § 365 (c), of an International application No. PCT/KR2024/095938, filed on Jul. 26, 2024, which is based on and claims the benefit of Korean Patent Application No. 10-2023-0098992 filed on Jul. 28, 2023, Korean Patent Application No. 10-2024-0023586 filed on Feb. 19, 2024, and Korean Patent Application No. 10-2024-0098856 filed on Jul. 25, 2024 in the Korean Intellectual Property Office, the disclosures of which are herein incorporated by reference in their entirety.

### TECHNICAL FIELD

The present disclosure relates to a method for treating hearing loss through auditory training.

### DESCRIPTION OF RELATED ART

As the population ages, the number of individuals with hearing impairments caused by presbycusis is increasing. Hearing specialists predict that this number will rise further due to the increase in average life expectancy. Additionally, hearing impairments can occur at any age due to congenital or acquired causes.

This has led to growing interest in assistive devices for hearing-impaired individuals (e.g., hearing aids, cochlear implants). In particular, there is increasing attention on cochlear implants for patients whose hearing does not improve even with the use of hearing aids. However, even after cochlear implantation, direct hearing is not immediately achievable, and rehabilitation training is essential.

Currently known rehabilitation training methods primarily involve repetitive listening to recorded sounds (e.g., words, short sentences) and solving related tasks. These methods often rely on monotonous and repetitive training, which can be inefficient and lead to user fatigue and boredom.

On the other hand, even individuals without significant hearing issues in daily life may seek to enhance their auditory functions for various reasons (e.g., improving musical abilities, developing talents in infants). Consequently, there is a growing demand for the development of user-friendly and effective auditory training (or rehabilitation) methods tailored to the individual characteristics of hearing-impaired individuals and/or users aiming to improve auditory function.

Existing rehabilitation training methods may include providing sounds corresponding to meaningful words or short sentences and evaluating whether users correctly recognize the sounds based on their responses. Using these conventional methods, hearing-impaired patients listen to the sounds and verify whether their recognition matches the correct answer. However, discrepancies between the actual sound and the user's recognition of the sound may occur.

In such cases, while the hearing-impaired individual can identify that their recognition differs from the actual sound, merely identifying this discrepancy may lead to prolonged rehabilitation periods. The patient would need to perform extended listening training to reduce the gap between their perception and the actual sound, which could result in time-intensive rehabilitation processes.

The present inventors, during their research to treat hearing loss, discovered that providing the actual sound along with its visual characteristics and/or receiving user input on the sound's features significantly enhances the effectiveness of hearing loss treatment. This discovery led to the development of the present invention.

### SUMMARY

The present invention has been devised to address the aforementioned issues and aims to provide a method for treating hearing loss through auditory training.

The technical objectives of the present invention are not limited to those mentioned above, and additional objectives not explicitly stated will be apparent to those skilled in the art from the following description.

To address the above-described technical challenges, the present invention provides a method for treating hearing loss through audiovisual interactive auditory training, comprising:
(i) providing a test sound having a first auditory pattern and instructing the patient to input a visual pattern corresponding to the test sound;
(ii) providing the patient with a first visual object having the first visual pattern when the patient inputs the first visual pattern, while simultaneously providing a first sound substantially synchronized with the detection timing of the pattern input; and
(iii) providing a second visual object having a second visual pattern corresponding to the first auditory pattern of the test sound.

The treatment method according to the present invention enables integrated training of auditory, visual, and motor feedback for hearing-impaired patients by providing auditory training sounds along with visual representations of sound characteristics and by receiving input from the patients regarding these characteristics. This method offers a more effective and intuitive learning environment compared to traditional voice-centered training methods, resulting in superior auditory improvement outcomes.

Moreover, the method allows for the identification of sound characteristics that the hearing-impaired patient struggles with, enabling tailored treatment for each individual. By focusing on these areas, the method facilitates more efficient improvement in auditory processing.

Additionally, the inclusion of both linguistic and non-linguistic training enhances music and language recognition abilities comprehensively, leading to effective rehabilitation across various areas, such as improved speech comprehension, better differentiation of everyday sounds, and enhanced music appreciation.

The advantages of the present invention are not limited to those mentioned above but also include other effects that can be clearly understood by those skilled in the art from the overall description of the specification, even if not explicitly stated.

### BRIEF DESCRIPTION OF THE DRAWINGS

A more complete appreciation of the disclosure and many of the attendant aspects thereof will be readily obtained as the same becomes better understood by reference to the following detailed description when considered in connection with the accompanying drawings, wherein:

FIGS. 1A to 1I illustrate an auditory training process using a sound sketchbook according to an embodiment.

FIG. 2 illustrates an auditory training process using a sound sketchbook based on information about vulnerable areas according to an embodiment.

FIGS. 3A and 3B illustrate a sound appreciation training process according to an embodiment.

FIGS. 4A to 4I illustrate a linguistic auditory training process according to an embodiment.

FIG. 5 illustrates a sub-home screen providing auditory training according to an embodiment.

FIG. 6 illustrates the interface of MAT software according to an embodiment.

FIG. 7 shows the results of a monosyllabic test for a pediatric cochlear implant patient.

FIG. 8 shows the results of a phoneme perception test for an adult cochlear implant patient.

FIG. 9 shows tone pattern recognition results before and after MAT training.

FIG. 10 shows the recognition results of monosyllabic words, consonants, and vowels before and after MAT training.

### DETAILED DESCRIPTION

Hereinafter, the preferred embodiments of the present invention will be described in detail with reference to the accompanying drawings. The advantages and features of the present invention, as well as the methods for achieving them, will become apparent by referring to the embodiments described below along with the attached drawings. However, the present invention is not limited to the embodiments presented hereinafter and can be implemented in various forms. These embodiments are provided merely to ensure the completeness of the description of the invention and to fully inform those skilled in the art of the scope of the invention, and the present invention is defined only by the scope of the claims. Throughout the specification, the same reference numerals refer to the same components.

Unless otherwise defined, all terms used in this specification (including technical and scientific terms) are used in a manner that would be commonly understood by those skilled in the art to which the invention pertains. Terms that are generally defined in dictionaries will not be interpreted in an excessively broad or narrow sense, unless explicitly defined otherwise. The terms used in this specification are intended to describe the embodiments and are not meant to limit the invention. In this specification, singular forms include plural forms unless specifically stated otherwise.

The terms “comprises” and/or “comprising” used in this specification do not exclude the presence or addition of one or more other components, steps, operations, and/or elements beyond those mentioned.

First, The present invention provides a method for treating hearing loss through audiovisual interactive auditory training, comprising the steps of:
(i) providing a test sound having a first auditory pattern and instructing the patient to input a visual pattern corresponding to the test sound; (ii) when the patient inputs the first visual pattern, providing the patient with a first visual object having the first visual pattern while providing a first sound substantially synchronized with the detection time of the pattern input; and (iii) providing a second visual object having a second visual pattern corresponding to the first auditory pattern of the test sound.

In the present invention, “hearing loss” refers to any condition in which hearing is impaired or lost.

The visual pattern can be input and provided through an interface for entering visual patterns, and the interface preferably includes a plurality of reference objects for association with the patient's input.

At this time, the reference objects are arranged in multiple rows, and the patient can input by connecting one of the plurality of first reference objects included in one of the rows to one of the plurality of second reference objects included in an adjacent row.

In one embodiment, based on the association of two or more reference objects among the plurality of reference objects with the first temporary patient input, a visual object associated with two or more reference objects related to the first temporary patient input is provided as a first visual object. Furthermore, based on the non-association of two or more reference objects among the plurality of reference objects with the second temporary patient input, the display of the visual object temporarily provided based on the trajectory of the second temporary patient input may be interrupted.

The number, density, and/or arrangement of the reference objects can be configured based on the patient's selection and/or the test difficulty.

The interface for inputting the visual pattern may include a first sub-interface for entering a projective visual pattern portion related to a first feature and a second sub-interface for entering a projective visual pattern portion related to a second feature. For example, the patient can input the projective visual pattern portion related to the first feature through the first sub-interface and input the projective visual pattern portion related to the second feature through the second sub-interface.

In another implementation, the interface may be configured to input information in three dimensions (first feature, second feature, time). For instance, when providing a VR environment, AR environment, or MR environment, it is possible to recognize the position of an input device (or a part of the patient's body) in three-dimensional space. Accordingly, a visual pattern defined in three dimensions may be recognized based on the trajectory of the input device (or a part of the patient's body) in three-dimensional space.

Meanwhile, based on the VR, AR, or MR environment, the aforementioned two-dimensional visual pattern can also be recognized.

In one embodiment, the lower limit for the test sound, the upper limit for the test sound, and/or the difference between the upper and lower limits can be set based on the patient's selection and/or the test difficulty.

In another embodiment, sound effects determined based on the patient's selection and/or the test difficulty can be applied to at least a portion of the test sound.

In one embodiment, the operation may further include providing background sound determined based on the patient's selection and/or the test difficulty along with at least a portion of the test sound.

In another embodiment, test sounds for the left and right ears in a stereo environment (or an earphone environment) can be provided. For example, test sounds can be offered only for the left ear, only for the right ear, or for both ears, thereby enabling test sounds tailored to multiple settings.

For instance, the patient may have impaired hearing in the left ear, in the right ear, or in both ears. Based on information about the ear corresponding to the patient's impaired hearing (e.g., cochlear implant surgery information, though not limited to this), the combination of test sound provision in the stereo environment can also be configured.

The provision of the sound may refer to outputting the sound through a speaker and/or transmitting data that causes sound output to an external speaker connected via wired or wireless means.

In step (ii), the operation of providing the first visual object may include providing at least a portion of the first visual object associated with the first position, the second position, and at least one intermediate position between the first and second positions, based on the detection of at least a portion of the patient input associated with the second position from the first position.

In one embodiment, based on at least a portion of the patient input being associated with the first position, the first part of the first sound, having at least one characteristic corresponding to the first position, is provided.

Furthermore, based on at least a portion of the patient input being associated with each of the at least one intermediate position, at least one intermediate part of the first sound, having at least one characteristic corresponding to each of the intermediate positions, is provided.

Finally, based on at least a portion of the patient input being associated with the second position, the second part of the first sound, having at least one characteristic corresponding to the second position, may be provided.

In one embodiment, at least a portion of the patient input may include input for specifying the first position and the second position and/or input for specifying the first position, at least one intermediate position, and the second position.

The hearing loss treatment method may further include: (iv) providing a comparison result of the first visual object and the second visual object.

Step (iv) can provide information about the patient's vulnerable areas identified based on the comparison result. These vulnerable areas may be expressed as specific frequency values or ranges.

In one embodiment, content for auditory training may be provided based on information about vulnerable areas specific to individual patients. Furthermore, as the patient's vulnerable areas change over time, content corresponding to the updated vulnerable areas may also be provided.

Steps (i) to (iii) can be repeated two or more times.

In such repetitions, the test sound for the next cycle may include the frequency values or ranges of the patient's vulnerable areas. Through these repetitions, sound characteristics that are challenging for the hearing-impaired patient can be identified, and sounds possessing the identified characteristics can be provided.

In step (i), the input can be performed through the patient's touch and/or continuous (or moving) touch (which may be referred to as dragging or clicking but is not limited to these). Preferably, the touch involves touching a provided screen.

Beyond simply stimulating visual and auditory senses, auditory training that incorporates motor feedback through direct touch and/or continuous (or moving) touch stimulates various regions of the brain. Activating multiple senses simultaneously can enhance neural plasticity in the brain, thereby improving learning outcomes. For example, it has been demonstrated in studies such as Literature ‘Shams, L., & Seitz, A. R. (2008). Benefits of multisensory learning. Trends in cognitive sciences, 12(11), 411-417’, ‘von Kriegstein, K. and Giraud, A. L. (2006) Implicit multisensory associations influence voice recognition. PLOS Biol. 4, e326’, ‘Hershenson, M. (1962). Reaction time as a measure of intersensory facilitation. J Exp Psychol, 63, 289-293’, ‘Shams, L., & Seitz, A. R. (2008). Benefits of multisensory learning. Trends in cognitive sciences, 12(11), 411-417’, ‘Nelson, W. T., Hettinger, L. J., Cunningham, J. A., et al. (1998). Effects of localized auditory information on visual target detection performance using a helmet-mounted display. Hum Factors, 40, 452-460’, ‘Diederich, A., & Colonius, H. (2004). Bimodal and trimodal multisensory enhancement: effects of stimulus onset and intensity on reaction time. Percept Psychophys, 66, 1388-1404’, ‘Sumby, W. H., & Pollack, I. (1954). Visual contribution to speech intelligibility in noise. J Acoust Soc Am, 26, 212-215’, ‘Lovelace, C. T., Stein, B. E., Wallace, M. T. (2003). An irrelevant light enhances auditory detection in humans: a psychophysical analysis of multisensory integration in stimulus detection. Brain Res Cogn Brain Res, 17, 447-453’, ‘Agelfors, E. (1996). A comparison between patients using cochlear implants and hearing aids. Part I: Results on speech tests. Quarterly Progress and Status Report’, ‘Desai, S., Stickney, G., Zeng, F. G. (2008). Auditory-visual speech perception in normal-hearing and cochlear-implant listeners. J Acoust Soc Am, 123, 428-440’, ‘Rouger, J., Fraysse, B., Deguine, O., et al. (2008). McGurk effects in cochlear-implanted deaf subjects. Brain Res, 1188, 87-99’, ‘Strelnikov, K., Rouger, J., Barone, P., et al. (2009). Role of speechreading in audiovisual interactions during the recovery of speech comprehension in deaf adults with cochlear implants. Scand J Psychol, 50, 437-444’, ‘Goh, W. D., Pisoni, D. B., Kirk, K. I., et al. (2001). Audio-visual perception of sinewave speech in an adult cochlear implant user: a case study. Ear Hear, 22, 412-419’, ‘Kaiser, A. R., Kirk, K. I., Lachs, L., et al. (2003). Talker and lexical effects on audiovisual word recognition by adults with cochlear implants. J Speech Lang Hear Res, 46, 390-404’, ‘Rouger, J., Lagleyre, S., Fraysse, B., et al. (2007). Evidence that cochlearimplanted deaf patients are better multisensory integrators. Proc Natl Acad Sci USA, 104, 7295-7300’, ‘Stevenson, R. A., Ghose, D., Fister, J. K., et al. (2014a). Identifying and quantifying multisensory integration: a tutorial review. Brain Topogr, 27, 707-730’ that stimuli across multiple domains are more effective for learning compared to single-sensory stimuli. In particular, bidirectional approaches, rather than unidirectional ones, may be more effective for learning. Thus, integrating multiple senses can maximize the effectiveness of auditory training.

For example, patients with hearing impairment may have difficulty receiving accurate feedback through hearing alone. In such cases, explicit feedback may be achieved by utilizing other sensory modalities. Unlike traditional methods that rely on linguistic or symbolic approaches, the training method according to the embodiment can use feedback based on other sensory modalities for non-linguistic auditory content.

For instance, the relationship between high and low tones in hearing may be cognitively linked to up-and-down and/or left-and-right relationships in visual/spatial perception. Auditory ability training based on these sensory connections can therefore be enabled.

Additionally, the invention may further include a training step in which the patient listens to and appreciates auditory patterns of the provided sound, as well as visual objects that visualize the auditory patterns of the output sound.

The sound visualization training step may include providing sound with an auditory pattern characterized by features (e.g., frequency, timbre, etc., but not limited to these) that change or remain constant over time and providing objects with visual patterns that visualize the auditory patterns of the sound's features.

While listening to the sound with auditory patterns, the patient can view the objects with visual patterns, enabling a visual understanding of the auditory patterns. This mapping of the auditory experience to visual patterns allows users to train their hearing.

The sound visualization training step may be performed before or after steps (i) through (iii).

As described above, after (or before, as the sequence is not limited) providing at least one training program based on non-linguistic elements, at least one training program based on linguistic elements may be provided.

A training program based on linguistic elements can include, for example, programs associated with phonemes defined by language.

Specifically, as a training program based on linguistic elements, the method may further comprise a linguistic auditory training step comprising:
(a) providing a test syllable sound composed of one vowel and one consonant, and instructing the patient to input a syllable object corresponding to the test syllable sound;
(b) providing a first syllable sound substantially synchronized with the detection timing of the patient's input when the patient inputs a first syllable object; and
(c) providing a second syllable object corresponding to the test syllable sound.

In step (a), the syllable object may include a plurality of reference syllable objects for association with the patient's input.

Before step (a), a step of selecting a target phoneme from one vowel and one consonant may be further included. In this case, the plurality of syllable objects may include the selected target phoneme.

Simultaneously with or after step (c), the match between the first syllable object and the second syllable object may be visually presented.

In one embodiment, each syllable learned by the patient (e.g., a combination of consonant and vowel) may be displayed in the intersection of the corresponding consonant and vowel. Based on the training of a specific syllable by the patient, the intersection of the consonant/vowel of that syllable may be displayed. If no training has been performed for a specific syllable, the intersection of the consonant/vowel for that syllable may be shown as empty.

Meanwhile, displaying the syllable at the intersection is an example, and objects that replace the syllable could be shown to indicate the completion of training for that syllable, indicate that the performance was good, or indicate that the performance was poor. If a syllable selection on the phoneme board is detected, a corresponding audio sound for the selected syllable may be provided.

When the performance result is good, the corresponding syllable may be represented with a first attribute (where attributes may include, but are not limited to, color, opacity, saturation, brightness, etc.).

When the performance result is poor, the corresponding syllable may be presented with a property different from the first attribute.

If the first syllable object and the second syllable object do not match, a training step may be further included, where the first syllable sound and the second syllable sound are compared and/or repeatedly listened to.

In this case, the first syllable sound and the second syllable sound may be provided randomly or alternately, but the method is not limited.

Based on the match or mismatch result of the first and second syllable objects, information regarding the patient's vulnerable phonemes may be provided. This result may be provided simultaneously with or after step (c).

In one embodiment, the pronunciation of phonemes defined by language may be positioned on a coordinate system based on the primary and secondary formant frequencies. Accordingly, a relatively higher number of incorrect answers in a test for a specific phoneme may indicate that the user is vulnerable to that frequency range, and additional training programs based on this information can be set.

Steps (a) through (c) may be repeated two or more times.

During the repetition, the test syllable sound for the next cycle may include the patient's vulnerable phoneme, and the vulnerable phoneme may be information identified based on the match or mismatch result of the first and second syllable objects.

Additionally, the treatment method according to one embodiment may include the following steps:
(i) playing a first content expressed audibly;
(ii) instructing the patient to input a dynamic representation of the visually expressed content corresponding to the first content; and
(iii) when the dynamic representation is input, visually providing the second content corresponding to the dynamic representation, while also providing the third content expressed audibly corresponding to the second content.

The treatment method according to one embodiment may also include:
(i) providing a non-verbal program associated with at least two of the following: the first content expressed audibly, the second content expressed visually, and the dynamic feedback provided by the user corresponding to the first content; and
(ii) providing a verbal program associated with the fourth content expressed audibly, which corresponds to a first phoneme composed of a first vowel from a plurality of vowels in the first language and a first consonant from the plurality of vowels, and the fifth content expressed visually corresponding to the fourth content.

A treatment method according to one embodiment may include:
(i) implanting a cochlear implant in a patient;
(ii) activating the implanted cochlear implant;
(iii) providing a non-linguistic program associated with at least two or more of the following: a first content presented audibly, a second content presented visually, and dynamic feedback provided by the user corresponding to the first content; and/or providing a linguistic program associated with a fourth content presented audibly and a fifth content presented visually, the fourth content being related to a first phoneme composed of a first vowel and a first consonant selected from a plurality of vowels and consonants in a first language;
(iv) adjusting the electrode settings of the cochlear implant based on the performance results of the non-linguistic program and/or the linguistic program.

Here, the non-linguistic program and/or the linguistic program in step (iii) may be configured based on performance results, user characteristics, and/or settings defined by an administrator (e.g., a therapist).

A treatment method according to one embodiment may include:
(i) implanting a cochlear implant in a patient;
(ii) activating the implanted cochlear implant;
(iii) adjusting the electrodes of the cochlear implant;
(iv) providing a non-linguistic program associated with at least two or more of the following: a first content presented audibly, a second content presented visually, and dynamic feedback provided by the user corresponding to the first content; and/or providing a linguistic program associated with a fourth content presented audibly and a fifth content presented visually, the fourth content being related to a first phoneme composed of a first vowel and a first consonant selected from a plurality of vowels and consonants in a first language.

A treatment method according to another embodiment may include:
(i) providing auditory rehabilitation content via a speech processor;
(ii) during the delivery of sub-content constituting the content, providing a non-linguistic program associated with at least two or more of the following: a first content presented audibly, a second content presented visually, and dynamic feedback provided by the user corresponding to the first content; and/or providing a linguistic program associated with a fourth content presented audibly and a fifth content presented visually, the fourth content being related to a first phoneme composed of a first vowel and a first consonant selected from a plurality of vowels and consonants in a first language.

The non-linguistic program and/or the linguistic program in step (ii) may be configured based on the performance results of the content, user characteristics, and/or settings defined by an administrator (e.g., a therapist).

At least a portion of the content in step (i) may be configured based on the performance results of the non-linguistic program and/or the linguistic program.

In the present invention, hearing loss includes conductive hearing loss and sensorineural hearing loss.

Here, conductive hearing loss refers to hearing loss caused by ear diseases, where problems occur in the organs that transmit sound, such as the eardrum and ossicles. Sensorineural hearing loss refers to hearing loss that occurs when there are problems with the cochlea, the auditory nerve that transmits sound as electrical energy, and the brain, which is responsible for comprehensive functions such as sound discrimination and understanding. The causes of sensorineural hearing loss can include noise, medication, aging, trauma, etc., and can, for example, be ototoxic hearing loss. Ototoxic hearing loss refers to hearing loss caused by the administration of one or more drugs selected from the group consisting of ototoxic drugs such as gentamicin, streptomycin, kanamycin, neomycin, amikacin, tobramycin, netilmicin, dibekacin, sisomycin, livodomycin, cisplatin, carboplatin, and oxaliplatin.

Additionally, the hearing loss may preferably include noise-induced hearing loss, presbycusis, sudden hearing loss, diabetic neuropathy, ototoxic hearing loss, traumatic hearing loss, viral hearing loss, etc., but is not limited to the scope of hearing loss as long as it corresponds to a state of hearing degradation or loss.

In the present invention, the target patients for the hearing loss treatment method may be patients who use cochlear implants. A cochlear implant is an auditory aid device used for patients with hearing loss, applicable to both congenital hearing loss (hearing loss present from birth) and acquired hearing loss (e.g., due to infections, drug side effects, or accidents). Regardless of the cause of the hearing loss, it may be used when the patient experiences severe hearing loss and hearing aids or other assistive devices are insufficient for adequate hearing improvement.

The cochlear implant uses electrical signals to stimulate the auditory nerve and directly transmit sound to the brain. This bypasses the damaged eardrum and auditory cells, helping the brain recognize sound. By using this treatment method, it can help improve the auditory recognition ability of patients using cochlear implants.

The treatment method of the present invention may further include a step of administering at least one selected from the group consisting of steroids, antibiotics, diuretics, and vasodilators to the patient, and preferably, this step may be performed before (the sequence is not limited to) the step (i).

The steroid may be used for the treatment of inflammation or sudden hearing loss (sudden sensorineural hearing loss), and preferably may be prednisolone.

The antibiotic may be used for treating hearing loss caused by infections such as otitis media, and preferably may be a cephalosporin-class antibiotic or amoxicillin.

The diuretic may be used for treating hearing loss related to Meniere's disease by regulating the fluid balance in the body and alleviating symptoms, and preferably may be furosemide.

The vasodilator may be a drug that helps improve blood flow inside the ear, alleviating blood flow issues that cause hearing loss, and preferably may be naftopidil.

The steroid, antibiotic, diuretic, or vasodilator may be administered orally or transdermally. The dosage may vary depending on factors such as the patient's weight, age, gender, health status, diet, administration time, administration method, excretion rate, and severity of the disease, with the daily dosage typically ranging from 0.01 to 1000 mg/kg, and may be adjusted depending on the route of administration, severity, gender, weight, age, etc.

The treatment method of the present invention may also be used in combination with a medical device and/or software for hearing training. The medical device and/or software for hearing training may include, for example, cochlear implant electrodes, external speech processors, speech processing algorithms, and/or auditory-verbal training, but is not limited to these.

Below, specific embodiments and experimental examples of the present invention will be described.

### Example 1. Non-Verbal Hearing Loss Treatment Training

### 1-1. Sound Sketchbook Auditory Training

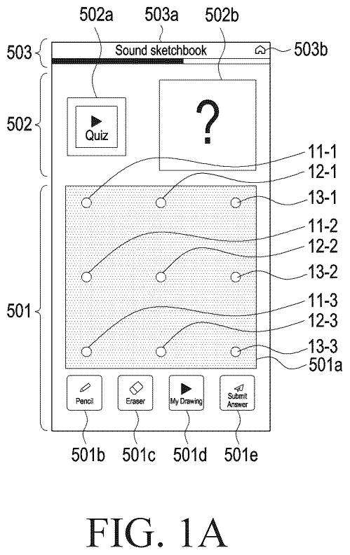

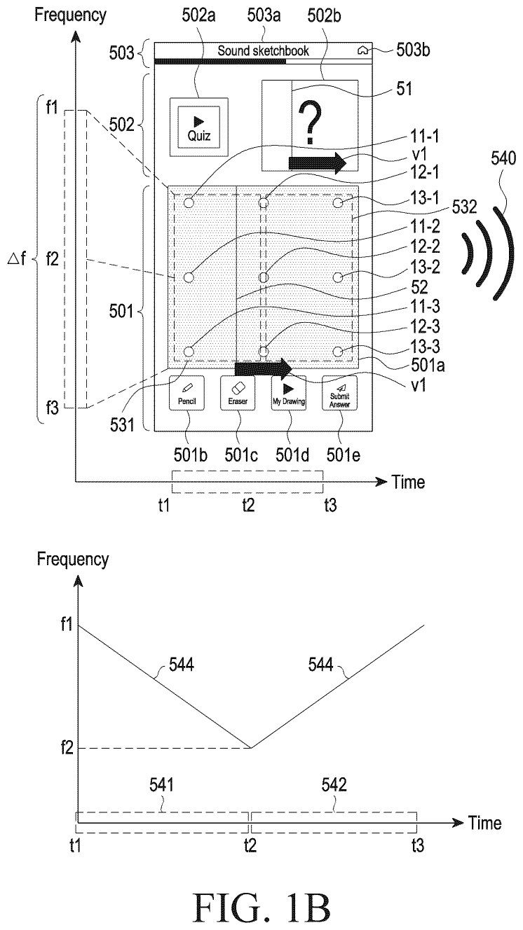

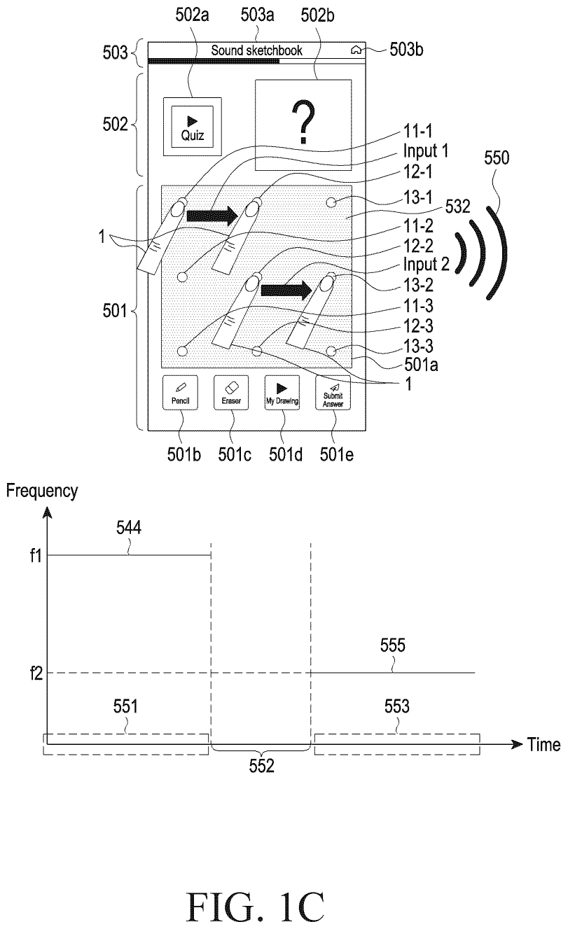

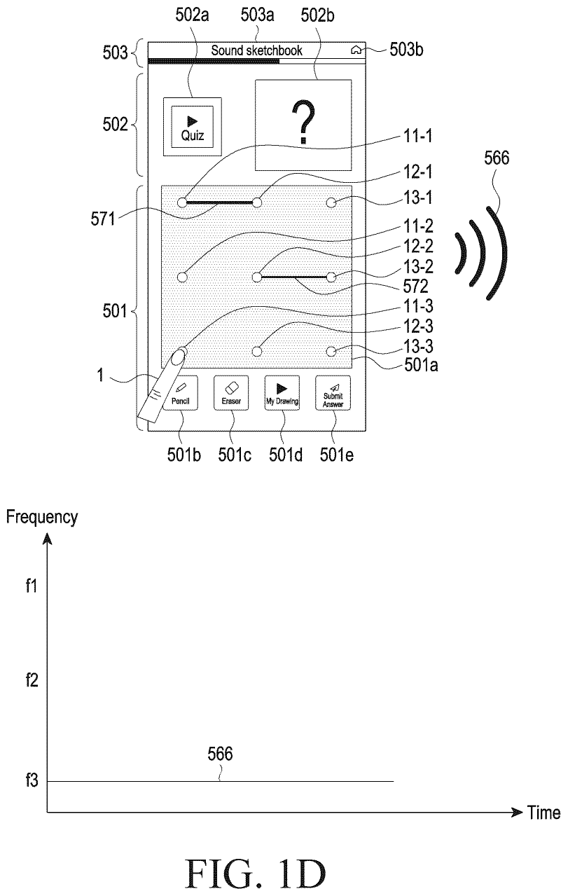

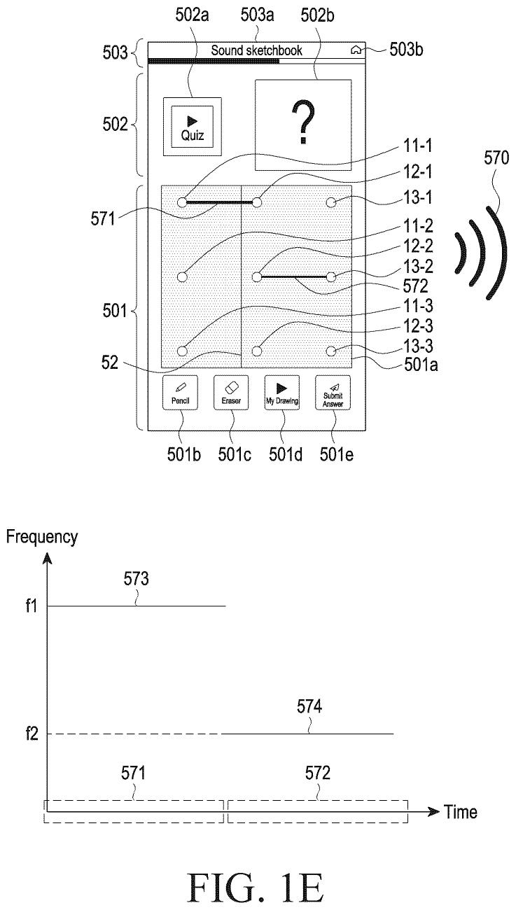

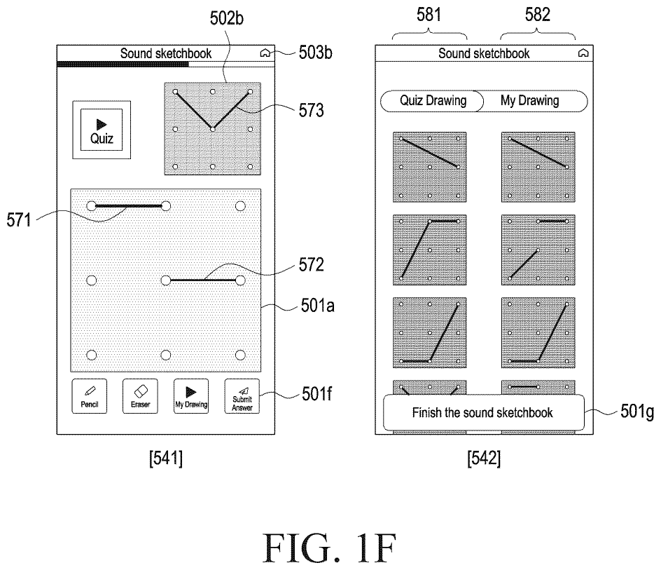

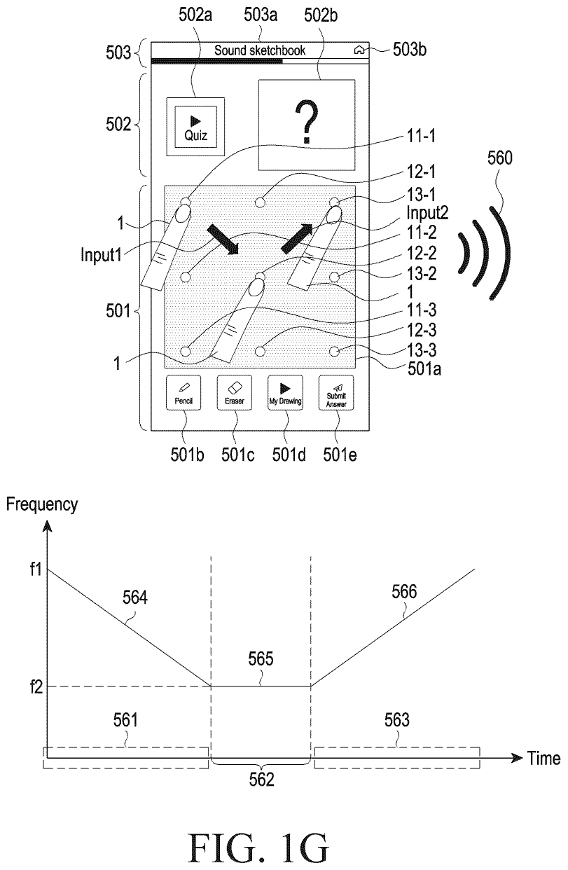

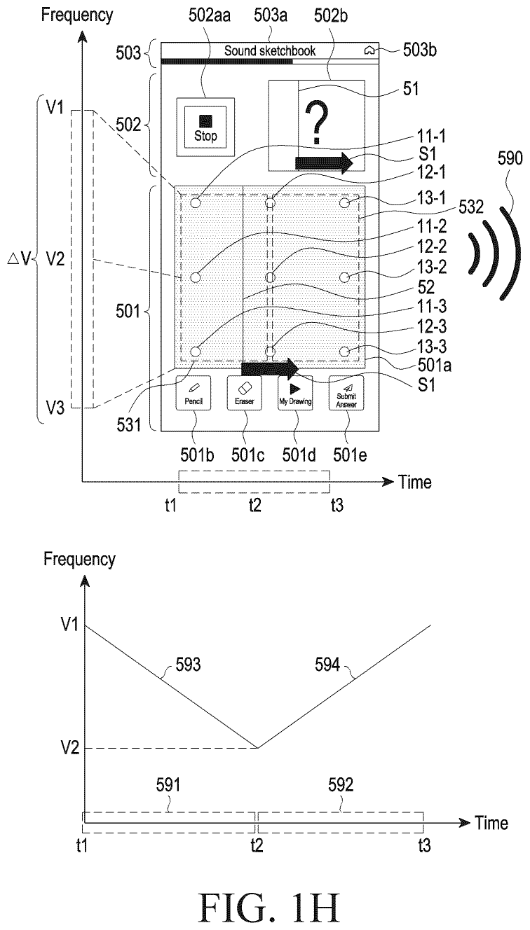

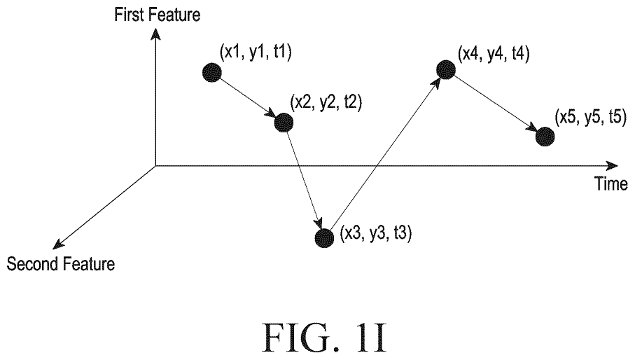

In one embodiment, an interface for inputting visual patterns is shown in FIG. 1A. As depicted in FIG. 1A, the interface may include multiple areas (501, 502, 503), though this is exemplary. The third area (503) may include a training name (503a) and a home object (503b) to return to the initial screen (311 or 312). The second area (502) may include a first play object (502a) that requests the provision (e.g., playback) of the first sound (or test sound), as well as a pattern display area (502b) for displaying the object corresponding to the first sound. The first area (501) may include a visual pattern input area (501a), a drawing object (e.g., pencil) (501b), an eraser object (e.g., eraser) (501c), a second play object (e.g., “my drawing”) (501d), and a correct answer check object (e.g., submit answer) (501e).

The visual pattern input area (501a) may include multiple first objects (11-1, 11-2, 11-3, 12-1, 12-2, 12-3, 13-1, 13-2, 13-3) for selecting one of the multiple designated values related to the characteristics of the test sound across multiple segments of the test sound. For example, the multiple first objects (11-1, 11-2, 11-3, 12-1, 12-2, 12-3, 13-1, 13-2, 13-3) may be matched to one of the designated values related to the sound characteristics. The multiple first objects may be arranged in a grid (e.g., 3 rows by 3 columns). In this case, the first objects included in each column may correspond to different values, while the first objects included in each row may correspond to the same value. For instance, the first objects (11-1, 12-1, 13-1) in the first row may correspond to a first value (e.g., a first frequency or first timbre), the first objects (11-2, 12-2, 13-2) in the second row may correspond to a second value (e.g., a second frequency or second timbre), and the first objects (11-3, 12-3, 13-3) in the third row may correspond to a third value (e.g., a third frequency or third timbre).

The first playback object (502a) can be set up to trigger the provision of the test sound when selected. In the correct visual pattern display area (502b), in response to the selection of the correct pattern confirmation object (501e) (or a request for the correct visual pattern), an object corresponding to the correct visual pattern, which matches the attributes of the test sound, may be displayed. However, before the selection of the correct pattern confirmation object (501e) (or the request for the correct visual pattern) is confirmed, a preview-prevention object (e.g., a question mark) may be displayed in the correct visual pattern display area (502b), as shown in FIG. 1A, for example. The placement of the correct visual pattern display area (502b) in a different region from the visual pattern input area (501a), as shown in FIG. 1A, is just an example. In another implementation, the interface may be designed not to include the correct visual pattern display area (502b) and may only include the visual pattern input area (501a). In this case, the object with the correct visual pattern may be displayed in the visual pattern input area (501a) together with the object containing the visual pattern entered by the patient, or may be displayed sequentially for the object containing the visual pattern entered by the patient, without limitation on the implementation method.

Based on the confirmation of the selection of the first playback object (502a), the test sound (540) can be provided. Based on the start of the provision of the test sound (540), the stop playback object (502aa) may replace the first playback object (502a) and be displayed, but there is no limitation on this.

The test sound includes a plurality of parts provided sequentially over time, each of which can change or remain constant over time according to the first auditory pattern. The test sound can have at least one characteristic (e.g., frequency, timbre, overtone density, and/or volume, but not limited to these). At a given point in time, each of the at least some of the characteristics of the test sound can have one value. At a given point in time, each of the at least some of the characteristics of the test sound can have multiple values.

At least some of the characteristics of the test sound can be designed for auditory training.

As an example, as shown in FIG. 1B, the frequency of the first part (543) of the test sound (540) can change from f1 to f2 over the first time interval (541) from the first time point (11) to the second time point (12) (for example, a linear change, but not limited to this change pattern). For example, the frequency of the second part (544) of the test sound (540) can change from f2 to f1 over the second time interval (542) from the second time point (12) to the third time point (13). Meanwhile, the frequency is simply an exemplary characteristic, and there is no limitation on the types of characteristics. Additionally, as shown in FIG. 1B, while the frequency characteristic is being changed, it is understood that some characteristics may remain constant over some time intervals.

According to the provision of the test sound (540), a first indicator (51) for visually indicating the playback position of the test sound (540) can be expressed, for example, as moving at a speed of v1 on the pattern display area (502b). The speed (v1) can, for example, be set according to the difficulty level of the test. For example, the horizontal length of the pattern display area (502b) can correspond to the entire time intervals (541, 542) during which the test sound (540) is provided.

For example, at the point in time when the provision of the test sound (540) is initiated, the first indicator (51) can be expressed as moving from the left side of the pattern display area (502b). Over time, as the test sound (540) is provided, the first indicator (51) can be expressed as moving to the right, in proportion to the accumulated time of the test sound (540) provided. At the third time point (13), the first indicator (51) can be expressed as reaching the right side of the pattern display area (502b).

For example, the second indicator (52) can be expressed as moving at the same speed (v1) as the first indicator (51) during the provision of the test sound (540). For instance, the horizontal length of the visual pattern input area (501a) can correspond to the entire time intervals (541, 542) during which the test sound (540) is provided.

For example, the first portion (531) of the visual pattern input area (501a) can correspond to the time interval (541), and the second portion (532) can correspond to the time interval (542).

For example, at the point in time when the provision of the test sound (540) is initiated, the second indicator (52) can be expressed as moving from the left side of the visual pattern input area (501a). Over time, as the test sound (540) is provided, the second indicator (52) can be expressed as moving to the right, in proportion to the accumulated time of the test sound (540) provided.

At the third time point (13), the second indicator (52) can be expressed as reaching the right side of the visual pattern input area (501a). Accordingly, the patient can recognize which part of the visual pattern input area (501a) they should input an object corresponding to the visual pattern of the test sound (540) they are listening to.

The vertical direction of the visual pattern input area (501a) can correspond to, for example, the features of the sound. As shown in FIG. 1B, the vertical direction of the visual pattern input area (501a) can correspond to a frequency range from f1 to f3. For example, the top of the visual pattern input area (501a) (or the objects placed at the very top, such as 11-1, 12-1, 13-1) can correspond to the frequency f1, the center of the visual pattern input area (501a) (or the objects placed at the center, such as 11-2, 12-2, 13-2) can correspond to the frequency f2, and the bottom of the visual pattern input area (501a) (or the objects placed at the very bottom, such as 11-3, 12-3, 13-3) can correspond to the frequency f3.

The frequency boundaries (f1, f3) and/or the frequency range (Δf) can be predefined or set according to the training difficulty, but are not limited to this. As shown in FIG. 1B, the patient can prepare to input an object corresponding to the visual pattern of the test sound (540) on the visual pattern input area (501a) while listening to the test sound (540).

Through this interface, the patient can input the visual pattern corresponding to the test sound.

In one embodiment, as shown in FIG. 1C, the patient can use a finger (1) to input the visual pattern corresponding to the test sound (540) on the visual pattern input area (501a). For example, the patient can input the visual pattern while the object (501b) for inputting the visual pattern is activated, although there is no limitation. Meanwhile, when the object (501b) for inputting the visual pattern is activated, the object (501c) for deleting a previously input visual pattern may be deactivated, but this is merely an example. For example, as shown in FIG. 1C, the patient can input the first input (input1), such as dragging, from object (11-1) to object (12-1) during the first time interval (551). During the second time interval (552), the patient can release the finger (1) from object (12-1) and then move the finger (1) to touch object (12-2). During the third time interval (553), the patient can input the second input (input2) from object (12-2) to object (13-2).

Based on at least one of these inputs, sound (550) can be provided. For example, the first part (554) of sound (550) can correspond to the features of the first input (input1). The first input (input1) can be, for example, a rightward movement from object (11-1) to object (12-1), and thus the first part (554), consisting of sub-sections with the first frequency (f1) corresponding to object (11-1), the sub-sections corresponding to each point between objects (11-1) and (12-1), and the sub-section corresponding to object (12-1), can be provided during the first time interval (551). Afterward, during the second time interval (552), if no input is detected, the sound may not be output. For example, the second part (555) of sound (550) can correspond to the features of the second input (input2). The second input (input2) can be, for example, a rightward movement from object (12-2) to object (13-2), and thus the second part (555), consisting of sub-sections with the second frequency (f2) corresponding to object (12-2), the sub-sections corresponding to each point between objects (12-2) and (13-2), and the sub-section corresponding to object (13-2), can be provided during the third time interval (553).

As mentioned above, while the patient is listening to the sound (550) corresponding to the points (or the progression of the points) they are inputting, they can input the visual pattern corresponding to the test sound (540).

Based on the inputs (input1, input2) as shown in FIG. 1C, objects (571, 572) with the confirmed visual pattern can be displayed. However, even after the objects (571, 572) are displayed, the patient can continue to make additional inputs. For example, as shown in FIG. 1D, if the patient touches object (11-3) and maintains the touch, sound (566) corresponding to object (11-3) can be provided. Sound (566) can have the third frequency (f3) corresponding to object (11-3). If no event for displaying an object with a visual pattern is detected, the patient's input may not cause objects like visual patterns (571, 572) to be displayed.

For example, an event for displaying an object with a visual pattern could be the confirmation of the patient's input, designating at least two objects (11-1, 11-2, 11-3, 12-1, 12-2, 12-3, 13-1, 13-2, 13-3) as starting and ending points. However, this is just an example, and there is no limitation on the types of events for displaying objects with visual patterns.

The patient can select the second play object (501d) if they wish to listen to the sound corresponding to the object they created. As shown in FIG. 1E, based on the selection of the second play object (501d), the stop-play object (501dd) may replace the second play object (501d), though there is no limitation. Additionally, based on the selection of the second play object (501d), the sound (570) corresponding to the object with the visual pattern (571, 572) can be provided.

For example, the first part (573) of sound (570) can be provided during the first time period (571). The first part (573) of sound (570) corresponds to the first part (571) of the object. Since the first part (571) of the object corresponds to the first frequency (f1), the first part (573) of sound (570) can have the first frequency (f1). For example, the second part (574) of sound (570) can be provided during the second time period (572). The second part (574) of sound (570) corresponds to the second part (572) of the object with the visual pattern. Since the second part (572) of the object corresponds to the second frequency (f2), the second part (574) of sound (570) can have the second frequency (f2).

The length of the first time period (571) may, for example, be substantially the same as the length of the first time period (541) in FIG. 1B during which the first part (543) of the test sound (540) is provided, though this is not a limitation. Similarly, the length of the second time period (572) may be substantially the same as the length of the second time period (542) in FIG. 1B during which the second part (544) of the test sound (540) is provided, but again, this is not a limitation.

Thus, the patient can verify whether the visual pattern they created corresponds to the test sound (540). If the patient recognizes that the visual pattern they created does not correspond to the test sound (540), they can activate (e.g., touch) the erase object (501c), delete at least part of the visual pattern object (571, 572) that was displayed, and input another visual pattern object.

In FIG. 1G, an input corresponding to the correct answer object based on the test sound (540) can be confirmed. For example, as shown in FIG. 1G, the patient can use their finger (1) to input a visual pattern corresponding to the test sound (540) within the visual pattern input area (501a).

For instance, as shown in FIG. 1G, during the first time period (561), the patient can input a first input (input1), such as dragging from object (11-1) to object (12-2) (the movement type is not limited). During the second time period (562), the patient can maintain the touch on object (12-1) with their finger (1). During the third time period (563), the patient can input a second input (input2) by moving from object (12-2) to object (13-1).

Additionally, sound (560) can be provided based on at least one input. For example, the first part (564) of sound (560) can correspond to the first input (input1). The first input (input1) may involve moving from object (11-1) to object (12-2) in a diagonal downward direction. As a result, the first part (564) of sound (560) may include sub-parts corresponding to the first frequency (f1) for object (11-1), intermediate frequencies (ranging from f1 to f2) for the multiple points between objects (11-1) and (12-2), and the second frequency (f2) corresponding to object (12-2). This first part (564) can be provided during the first time period (561).

Later, during the second time period (552), based on the patient's maintained input on object (12-2), the second part (565) corresponding to the second frequency (f2) can be provided. The second part (565) of sound (560) can correspond to the patient's touch on object (12-2).

For the second input (input2), for example, the patient may move from object (12-2) to object (13-1) in an upward diagonal direction. This results in the third part (566) of sound (560) composed of sub-parts corresponding to the second frequency (f2) for object (12-2), intermediate frequencies (ranging from f2 to f1) for the multiple points between objects (12-2) and (13-1), and the first frequency (f1) for object (13-1). This third part (566) can be provided during the third time period (563).

Thus, the patient can listen to sound (560) corresponding to the points (or transitions between points) they are inputting, and can input the visual pattern object corresponding to the test sound (540).

FIG. 1H illustrates a sound test based on characteristics other than frequency, according to an example embodiment.

For instance, in the examples of FIGS. 1B to 1E, frequency could be set as the target feature for training among the various characteristics of the test sound. In contrast, in FIG. 1H, the volume is set as the target feature for training among the characteristics of the test sound.

In FIG. 1H, the vertical direction of the visual pattern input area (501a) corresponds to the volume range from V1 to V3. For example, the top part (or objects placed at the top) of the visual pattern input area (501a), such as objects (11-1, 12-1, 13-1), corresponds to volume V1. The middle part (or objects placed in the center) of the visual pattern input area (501a), such as objects (11-2, 12-2, 13-2), corresponds to volume V2. The bottom part (or objects placed at the bottom) of the visual pattern input area (501a), such as objects (11-3, 12-3, 13-3), corresponds to volume V3.

The volume boundary values (V1, V3) and/or the volume range (AV) can be set according to predefined specifications or training difficulty, but are not limited. Detailed explanations of this are provided later.

As shown in FIG. 1H, the patient can listen to parts (593, 594) of the test sound provided during time periods (591, 592) and prepare to input a visual pattern corresponding to the test sound into the visual pattern input area (501a).

Moreover, it should be understood that, in addition to characteristics that can be expressed as values like frequency or volume, all characteristics of sound can be utilized for auditory training without limitation.

FIG. 1I illustrates a multi-dimensional feature-based auditory training according to an embodiment.

In this example, multiple characteristics (e.g., a first feature and a second feature) of the test sound can be used for auditory training.

For instance, the test sound may include a portion at the first time point (t1) that has a first feature with a value of x1 and a second feature with a value of y1. Between the first time point (t1) and the second time point (t2), the test sound may include a portion where the first feature changes from x1 to x2 and the second feature changes from y1 to y2.

Similarly, between the second time point (12) and the third time point (13), the test sound may include a portion where the first feature changes from x2 to x3 and the second feature changes from y2 to y3. Between the third time point (t3) and the fourth time point (14), the test sound may include a portion where the first feature changes from x3 to x4 and the second feature changes from y3 to y4.

Finally, between the fourth time point (14) and the fifth time point (15), the test sound may include a portion where the first feature changes from x4 to x5 and the second feature changes from y4 to y5.

In this way, multiple features of the sound can be varied over time to train the auditory system in a multi-dimensional manner.

### 1-2. Auditory Training Based on Vulnerable Areas

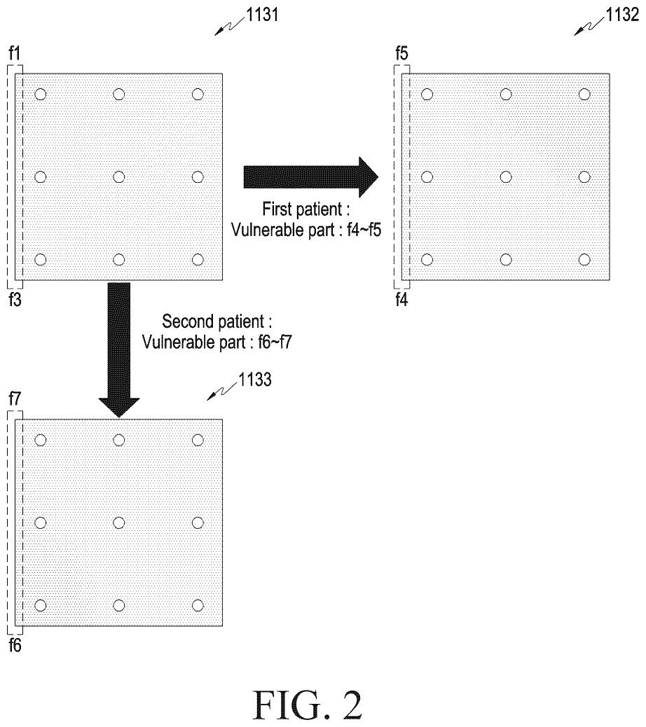

As an example, content for auditory training can be provided based on information about the vulnerable areas of each patient. For example, as shown in FIG. 2, based on the results of using content (1131) for auditory training based on the first frequency range (f1 to f3), the first vulnerable area can be identified as the frequency range from f4 to f5, and the second vulnerable area as the frequency range from f6 to f7.

Based on the first vulnerable area, content (1132) for auditory training based on the second frequency range (f4 to f5) can be provided for the first patient. Based on the second vulnerable area, content (1133) for auditory training based on the third frequency range (f6 to f7) can be provided for the first patient. For example, depending on the changes in the patient's vulnerable areas over time, content corresponding to the changed vulnerable areas can also be provided.

### 1-3. Sound Image Appreciation Training

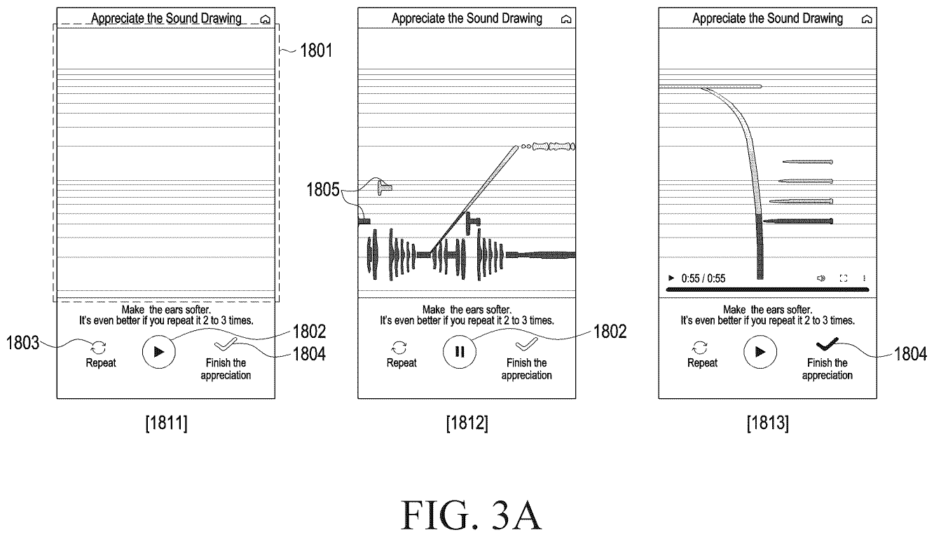

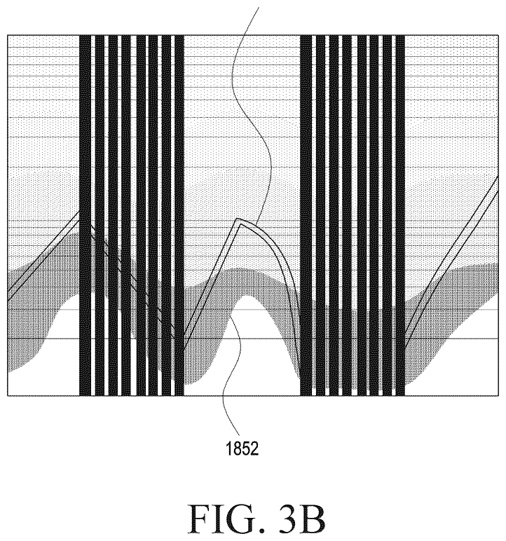

As shown in FIGS. 3A and 3B, content for auditory training based on sound image appreciation can be provided.

As illustrated in FIG. 3A, screens (1811, 1812, 1813) for sound image appreciation training can be provided. The content for this training includes providing a sound with an auditory pattern in which characteristics (e.g., frequency, timbre, etc., though not limited to these) change or remain consistent over time, and providing a visual object with a visual pattern that visualizes the auditory pattern of the sound characteristics outputted.

For example, when sound image appreciation training is requested, the sound image appreciation training screen (1811) can be provided. The screen (1811) includes a visual area (1801) that displays visual objects, a 5th play object (1802) that plays the designated training sound, a repeat object (1803) that repeats the training sound, and an end training object (1804) to finish the sound image appreciation training.

When the 5th play object (1802) is selected, the designated training sound can be provided (e.g., played or output). The screen (1812) may include a visual object (1805) corresponding to the designated training sound, which is displayed in the visual area (1801). At this point, the 5th play object (1802) may change into a stop object (1806). The representation of the visual object (1805) can be synchronized with the provision of the training sound. For example, as the training sound is provided over time, the visual object (1805) can be gradually displayed, with animation effects provided. However, there are no limitations to the synchronization method, as one skilled in the art would understand.

When the playback of the training sound is completed, the “Finish Appreciation” object (1804) can be activated. At this point, the stop object (1806) can change (or be restored) to the 5th play object (1802). Afterward, the patient can either select the “Finish Appreciation” object (1804) to end the sound image appreciation training, select the 5th play object (1802) to repeat the training, or select the repeat object (1803) to repeat the training for a specified number of times (e.g., 5 times) or until the stop object (1806) is selected.

While listening to the auditory pattern of the sound, the patient can view the visual pattern of the object, helping them understand the auditory pattern visually. By mapping the auditory result to the visual object, the patient's hearing can be trained.

Additionally, as shown in FIG. 3B, specified effects and/or background sounds can be provided along with the training sound. For example, a visual object (1851) with a visual pattern corresponding to the training sound can be displayed in synchronization with the sound. An object (1852) corresponding to a specified effect (e.g., disperse effect) can also be shown. Though not shown, an object corresponding to background sound can be displayed for intermittent pauses in the provided sound. By visually observing not only the training sound but also the effects and/or background sounds applied to the training sound, patients can undergo more advanced auditory training.

### Example 2: Auditory Training for Speech Impairment

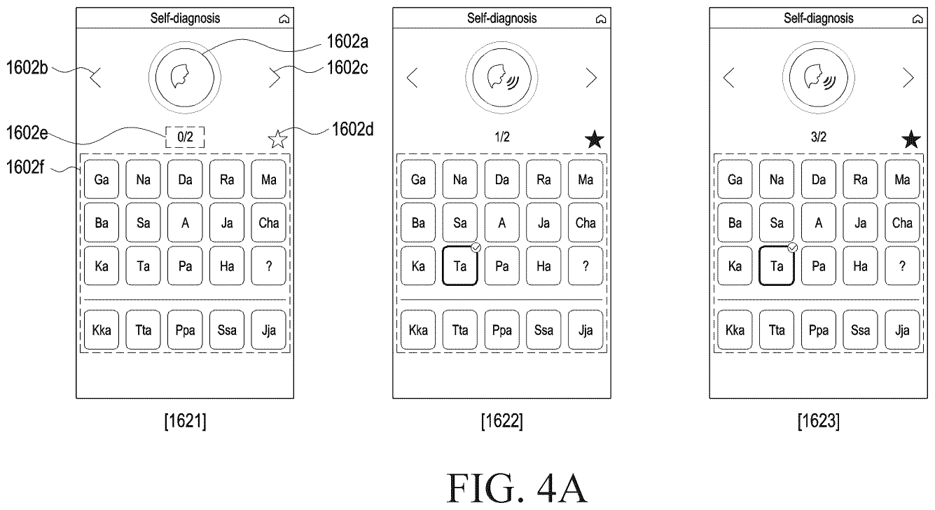

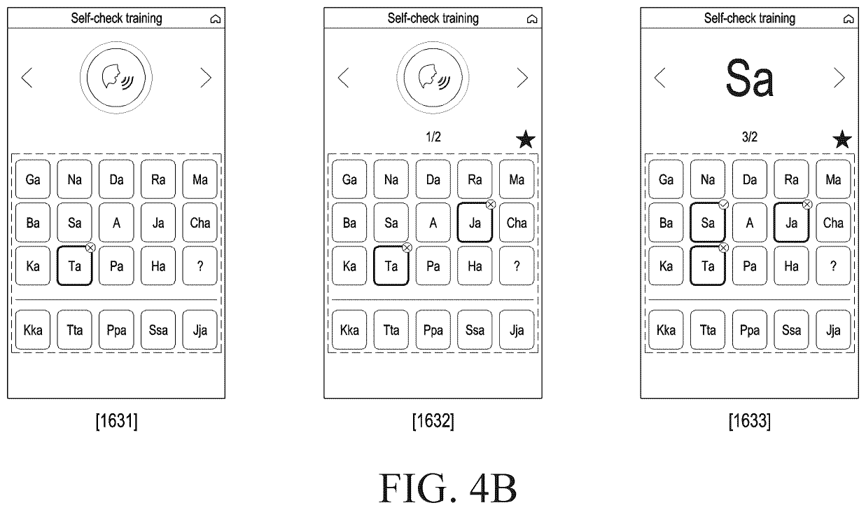

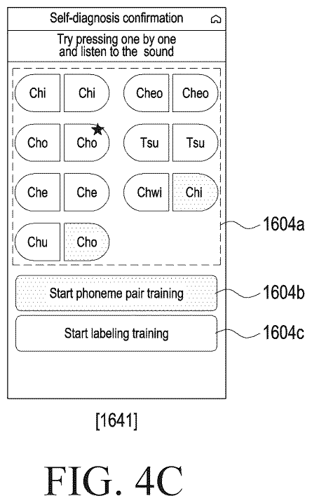

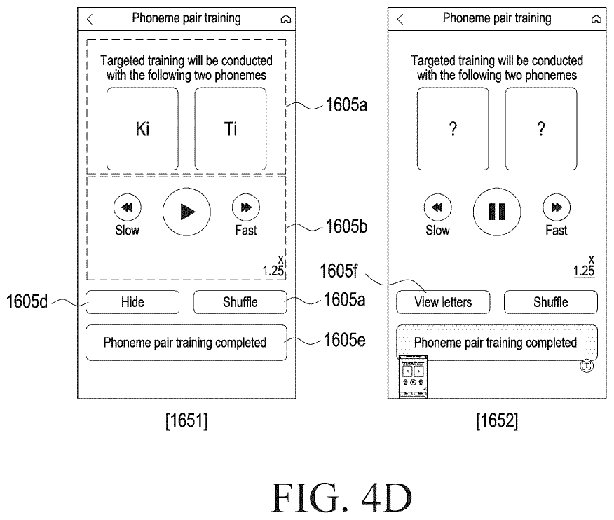

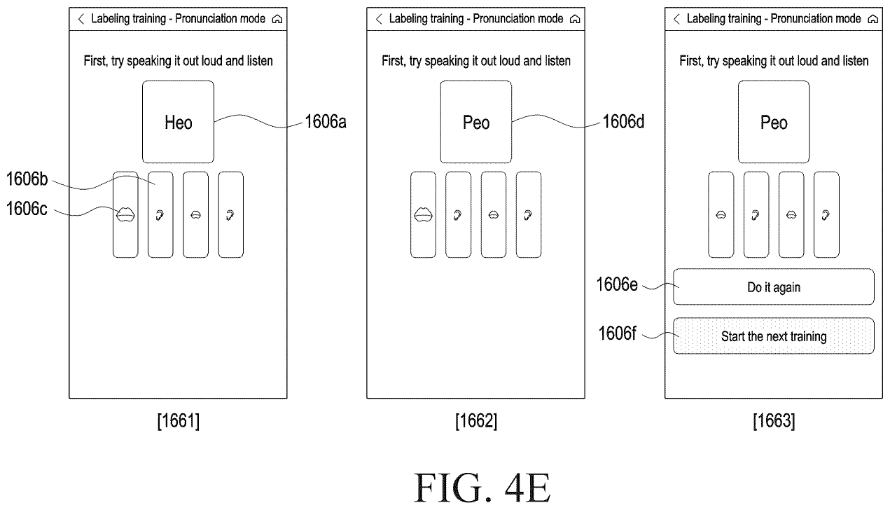

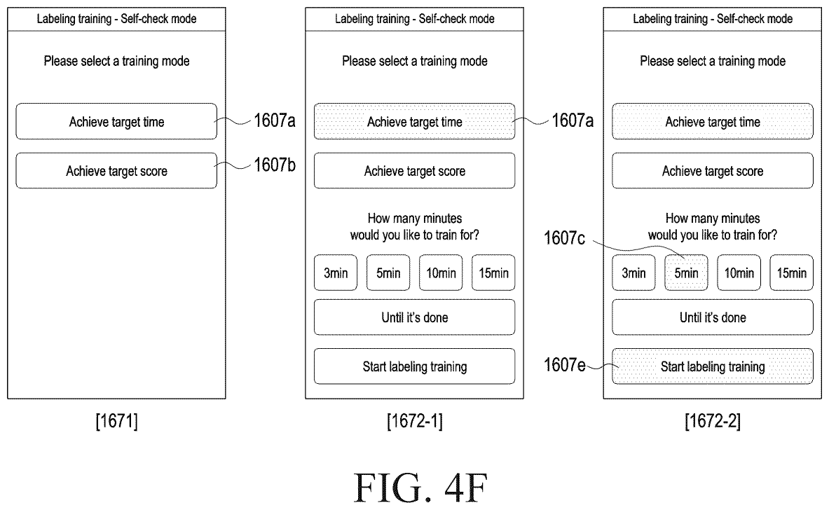

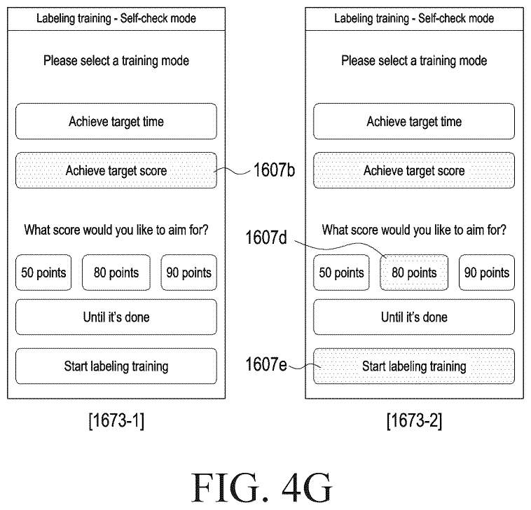

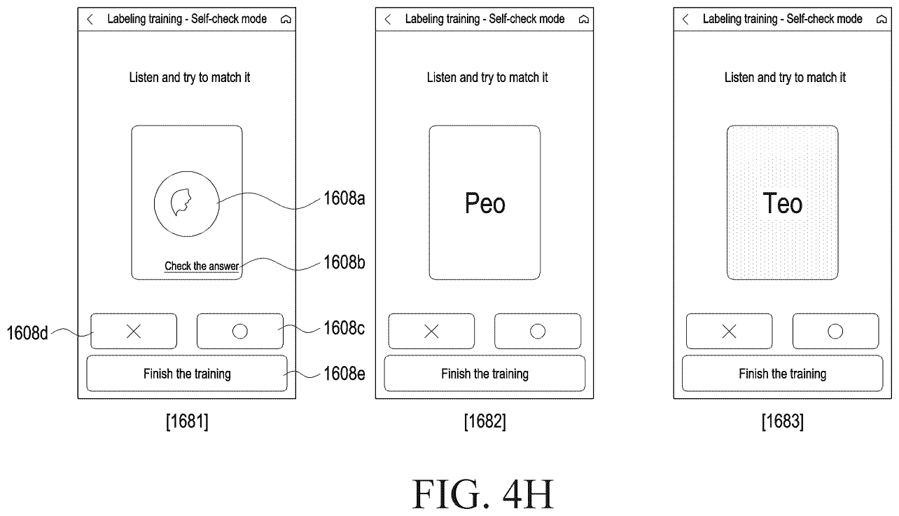

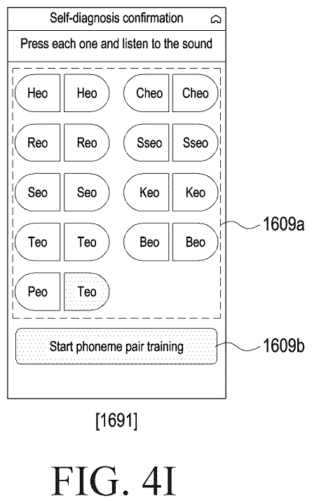

As an example, a screen for auditory training related to speech impairment is shown in FIGS. 3A and 3B. As shown in FIGS. 4A and 4B, at least some of the screens (1621, 1622, 1623, 1631, 1632, 1633) can be provided. These screens may include the following components: a 3rd play object (1602a) to request the playback of a problem (e.g., one of the syllables formed by the combination of consonants and vowels to be trained), a 1st move object (1602b) to move to the previous problem, a 2nd move object (1602c) to move to the next problem, and a registration object (1602d) to register the problem of interest.

For example, the first self-diagnosis training screen may include a count display area (602e) that shows the number of times each problem is listened to and a syllable display area (1602f) that shows multiple syllables that can be selected.

In the training, after listening to the provided problem, the patient may select one of the multiple syllables shown in the syllable display area (1602f), but there is no limitation on how the selection process works. For example, when the 3rd play object (1602a) is selected, the corresponding syllable can be provided through an audio output device. Referring to screen (1622), one of the syllables displayed in the syllable display area (1602f) can be confirmed as being selected by the patient. Referring to screens (1622, 1623), the listening count in the count display area (602e) may increase each time the 3rd play object (602a) is selected.

For example, referring to screens (1631, 1632), when an incorrect answer is entered, a visual notification may be provided to indicate the mistake (e.g., changing the background to a first color, such as red, and/or displaying a designated symbol in the first color, such as an “x” on one side).

For example, referring to screen (1633), when the correct answer is selected, a visual notification may be provided to indicate the correctness (e.g., changing the background to a second color, such as green, displaying a designated symbol in the second color, such as a “V” on one side, and/or changing the 3rd play object (1602a) to the correct syllable or replacing it).

For example, after completing the training up to the last problem, when the 2nd move object (1602c) to request the next problem is selected, a self-diagnosis result screen may be provided. The first self-diagnosis result screen (1641), as shown in FIG. 4C, may include a result display area (1604a) that displays the syllable (or character) corresponding to the problem and the syllable selected by the patient as a pair (referred to as a phoneme pair).

For example, the syllable corresponding to the problem is displayed on the left side, and the syllable selected by the patient is displayed on the right side, showing a phoneme pair. If an incorrect answer is identified, the background color of other syllables may be displayed in a different color.

For example, the first self-diagnosis result screen (1641) may include a phoneme pair training object (1604b) for incorrect problems and a labeling training object (1604c). At this point, the labeling training object (1604c) is displayed in a deactivated state, and can be activated after the phoneme pair training is completed. For instance, if there are no incorrect answers in the previous training, the phoneme pair training object (1604b) may be deactivated (or not displayed), and only the labeling training object (1604c) may be activated.

For example, when the phoneme pair training object (1604b) is selected, a phoneme pair training screen may be provided. Phoneme pair training is designed to help recognize the differences by repeatedly listening to incorrect phoneme pairs. For instance, when the phoneme pair training object (1604b) is selected, as shown in the screen (1651) of FIG. 4D, the phoneme pair training screen may be provided. The phoneme pair training screen may include a phoneme pair display area (1605a) for providing the phoneme pair information to be trained, a playback control area (1605b) for controlling the playback speed, a shuffle object (1605c) for randomly changing the order of the phoneme pairs, a hide object (1605d), and/or a training end object (1605e).

After a specified period of time has passed, as shown in the screen (1652) of FIG. 4D, the phoneme pairs to be trained may be hidden (e.g., marked with a question mark), and the order of the phoneme pairs may be randomly changed, with each phoneme's corresponding sound being provided alternately. At this point, the hide object (1605d) may be replaced with a letter view object (1605f) that displays the hidden letters. After sufficiently listening to the alternating phoneme pairs, the patient can select the letter view object (1605f) to confirm whether they have heard it correctly. In this case, the letter view object (1605f) may be replaced with the hide object (1605d).

For example, when the phoneme pair training is completed, the labeling training object (1604c) may be activated. When the labeling training object (1604c) is selected, a labeling training screen may be provided. Labeling training may include both articulation training and self-diagnosis training. Articulation training is a practice that repeats speaking (or pronouncing) and listening (or hearing). For example, as shown in screen (1661) of FIG. 4E, the syllable to be trained (e.g., “heo”) (1606a) may be displayed on one side, and the speaking icon (1606b) and listening icon (1606c) may alternate between being activated. When the listening icon (1606c) is activated, the sound of the syllable may be provided. In some embodiments, when the speaking icon (1606b) is activated, the patient's voice may be recorded.

For example, after the specified number of repetitions for articulation training (e.g., 2 times) has passed, a training screen for the next syllable (e.g., “peo”) (1606d) may be provided, as shown in screen (1662). Once all training is completed, an articulation training result screen may be provided, as shown in screen (1663). The articulation training result screen may include a re-learning object (1606e) for re-training articulation and/or a next training object (1606e) to exit articulation training and move to the next training.

For example, if the next training object (1606e) is selected, the self-diagnosis training of the labeling training may be provided. For example, when the next training object (1606e) is selected, a screen (1671) for selecting the training mode of the self-diagnosis training of labeling training may be provided, as shown in FIG. 4F. The self-diagnosis training of labeling training may include a target time training mode (1607a), where the patient trains for a specified amount of time, and a target score training mode (1607b), where the patient trains until a target score is reached. When one of these modes is selected by the patient, the target is set, and the self-diagnosis training of labeling training may begin.

For example, as shown in screens (672-1, 672-2), the target time training mode (1607a) may be selected, and a target time (e.g., 5 minutes) (1607c) may be chosen. Alternatively, as shown in screens (1673-1, 1673-2) in FIG. 4G, the target score training mode (1607b) may be selected, and a target score (e.g., 80 points) (1607d) may be chosen. Once the target time (1607c) or target score (1607d) is set, the training start object (1607e) may become active.

Once the target time (1607c) or target score (1607d) is set and the training start object (1607e) is selected, the self-diagnosis training screen (1681) of labeling training may be provided, as shown in FIG. 4H. The self-diagnosis training screen (1681) of labeling training may include a fourth playback object (1608a), a correct answer display object (1608b), a correct answer object (1608c), an incorrect answer object (1608d), and/or a training exit object (1608e). If the fourth playback object (1608a) is selected, a sound corresponding to the trained syllable may be played. The patient may then attempt to estimate the syllable corresponding to the heard sound.

If the correct answer display object (1608b) is selected, as shown in screen (1682), the fourth playback object (1608a) may change (or be replaced) to the syllable (or character) corresponding to the sound. For example, the deactivated correct answer object (1608c) and the incorrect answer object (1608d) may be activated to allow the patient to select them.

The patient can compare the syllable they estimated with the correct answer and choose either the correct answer object (1608c) or the incorrect answer object (1608d). For example, if the patient believes their estimated syllable matches the correct answer, they can select the correct answer object (1608c). If their estimation is incorrect, they can select the incorrect answer object (1608d).

Once either the correct answer object (1608c) or the incorrect answer object (1608d) is selected, the next question screen may be provided. Specifically, if the correct answer object (1608c) is selected, as shown in screen (1683), the corresponding sound is played once, and the correct answer is visually indicated (e.g., the border of the area displaying the syllable is changed to the first color), after which the next question screen is provided. Conversely, if the incorrect answer object (1608d) is selected, the corresponding sound is played the designated number of times (e.g., 3 times), and the incorrect answer is visually indicated (e.g., the border of the area displaying the syllable flashes with the second color), followed by the next question screen.

When the patient reaches the target time (1607c) or target score (1607d), the inactive training end object (1608e) may be activated, making it selectable. However, even after reaching the target time or target score, the patient may choose not to select the training end object (1608e) and continue with further training.

On the other hand, when the training end object (1608e) is selected, a result screen (1691) as shown in FIG. 4I can be provided. The result screen (1691) is similar to the first self-diagnosis result screen (1641) shown in FIG. 4C and may include a result display area (1609a) that shows the trained phoneme pairs. Since both the phoneme pair training and labeling training have been completed in the result screen (1641), instead of the phoneme pair training object (1604b) and labeling training object (1604c), the next training object (e.g., the second self-diagnosis training) (1609b) may be included.

When the next training object (1609b) is selected, the second self-diagnosis training may be provided. In some embodiments, when the next training object (1609b) is selected, the home screen may be displayed, or the object corresponding to the completed training may be shown in an activated state.

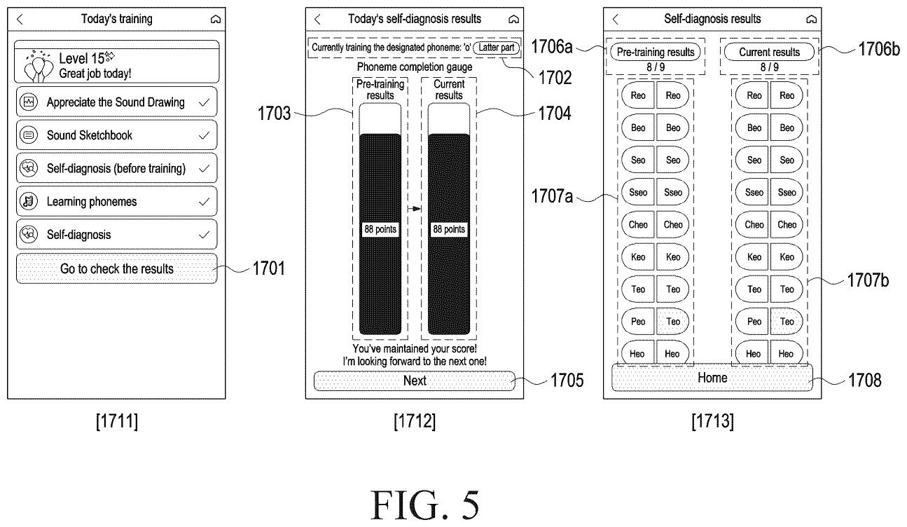

Once the second self-diagnosis training is completed, a sub-home screen (1711) can be provided or the system can return to the sub-home screen. As an example, as shown in FIG. 5, the sub-home screen (1711) may display objects corresponding to all completed trainings in an activated state. Additionally, the sub-home screen (1711) may include a result confirmation object (1701). The result confirmation object (1701) may be displayed based on the completion of all the trainings represented on the sub-home screen (1711).

When the result confirmation object (1701) is selected, the first result screen (1712) can be provided. The first result screen (1712) may include the phoneme information (1702) being trained, the first self-diagnosis training result (1703), the second self-diagnosis training result (1704), and a next object (1705) to request the second result screen. In the first result screen, the first self-diagnosis training result (1703) and the second self-diagnosis training result (1704) may be provided, for example, using bar graphs, though this is not limited to these methods.

When the next object (1705) is selected, the second result screen (713) can be provided. The second result screen (713) may include the accuracy of the first self-diagnosis training (1706a), the accuracy of the second self-diagnosis training (1706b), the phoneme pairs trained in the first self-diagnosis training (1707a), the phoneme pairs trained in the second self-diagnosis training (1707b), and a home object (1708) to return to the home screen.

### Example 3. Multimodal Acoustic Therapy (MAT)

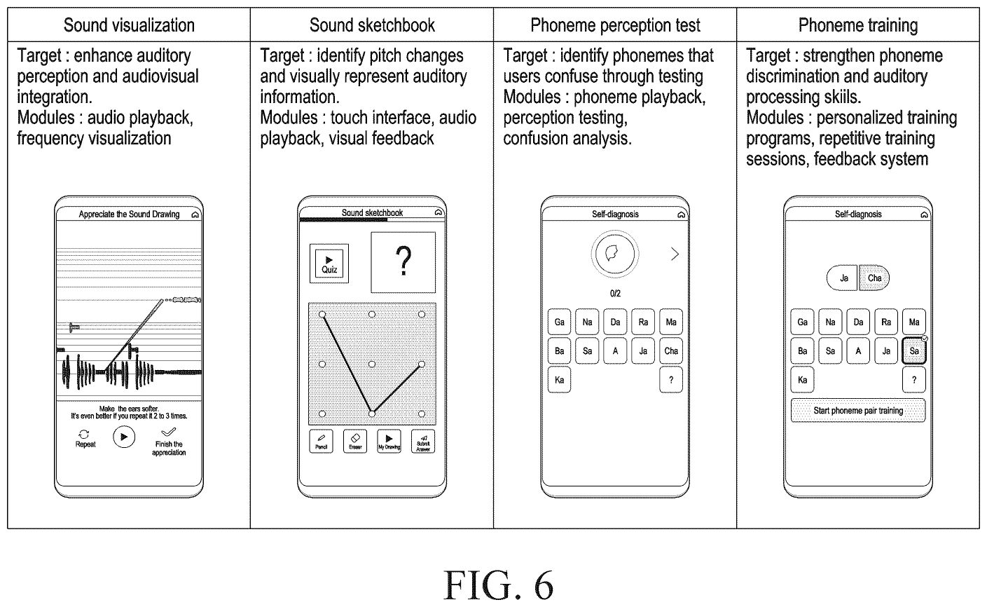

MAT (Multimodal Acoustic Therapy) utilizes smartphone-based technology to provide real-time multisensory content and/or feedback by integrating at least two of the auditory, visual, and motor-based inputs, but this is not limited. Some of the programs in FIG. 6 may be based on MAT. For example, Sound Visualization, as described in embodiments 1-3, may be a program based on visual and auditory inputs. Similarly, Sound Sketchbook, as described in embodiments 1-1 and 1-2, may be a program based on visual, auditory, and motor inputs. Phoneme Perception Test and Phoneme Training, as described in embodiment 2, may be programs based on visual and auditory inputs. The phonemes used in these programs may, for example, be meaningless in nature.

The example in FIG. 6 illustrates the use of non-verbal programs such as Sound Visualization and Sound Sketchbook, and verbal programs such as Phoneme Perception Test and Phoneme Training, but this is not restrictive.

These programs combine verbal and non-verbal sound training, and each program is as follows.
Visual Scene Appreciation: As described in Examples 1-3, various sounds are provided to the subject along with their corresponding visual representations. Frequency changes are mapped to vertical movements in the spatial dimension, enabling participants to visually perceive pitch patterns.
Sound Sketchbook: As described in Examples 1-1 and 1-2, the subject participates in an auditory dictation activity by drawing the perceived sound patterns on a touchscreen, combining auditory information with visual and motor information.
Phoneme Recognition Test: As described in Example 2, the subject conducts self-assessment by listening to phonemes and selecting the correct answers.
Acoustic Phoneme Training: As described in Example 2, the subject practices phonemes through intensive and repetitive training.

### Experimental Example 1. Monosyllabic Test for Pediatric Cochlear Implant Patients

A teenage male patient, referred to as Y, who underwent cochlear implant surgery for congenital hearing loss, was selected as the subject. The auditory training method described in Example 1 was delivered through a smartphone application. During the training session, the patient freely interacted with the application by touching the screen to generate sounds and learn sound patterns independently. At the initial use, the researcher provided guidance on how to use the application. The training was designed to help cochlear implant users modify sound characteristics through finger movements, recognize the changes, and clearly understand pitch variations through visual stimuli.

Before starting the first training session, a monosyllabic pre-test was conducted. This test, widely used in clinical settings to assess speech perception, required the patient to listen to monosyllabic words consisting of an initial consonant, a vowel, and a final consonant, and then identify the perceived syllable. After the pre-test, the cochlear implant user underwent a 30-minute training session using the application.

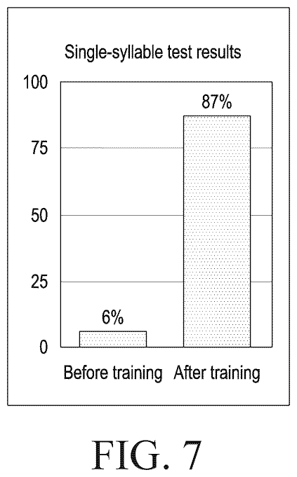

Following the session, the same direct monosyllabic test was administered in a randomized order to evaluate the effect of the training. The results are presented in Table 1 and FIG. 7.

As shown in Table 1 and FIG. 7, the accuracy rate improved significantly from 6% during the pre-intervention test to 87% after the first training session. To further explore the intervention's effects, the cochlear implant user continued rehabilitation training at home for seven days. Subsequent testing conducted in the same location and in a randomized order showed an accuracy rate improvement to 100%. Additionally, the caregiver of the cochlear implant user reported noticeable changes in daily life during the week of training. For instance, the user exhibited improved auditory perception, being able to detect and understand the whispered voices of family members (soft sounds) that had previously gone unnoticed.

TABLE 1

| | Pre- | Post-Treatment | Post-Treatment |
|---|---|---|---|
| | Intervention | Response 1 | Response |
| Monosyllabic | Response | (30-Minute | 2 (7-Day |
| Words | (baseline) | Intervention) | Intervention) |
| kal | X | ◯ | ◯ |
| bam | X | ◯ | ◯ |
| kkot | X | ◯ | ◯ |
| ttam | X | X | ◯ |
| chong | X | ◯ | ◯ |
| ppul | X | ◯ | ◯ |
| dool | X | ◯ | ◯ |
| cho | X | ◯ | ◯ |
| bang | X | ◯ | ◯ |
| sam | X | ◯ | ◯ |
| gam | X | ◯ | ◯ |
| hok | X | ◯ | ◯ |
| ok | X | ◯ | ◯ |
| ot | X | ◯ | ◯ |
| chang | ◯ | X | ◯ |
| Correct Rate | 6% | 87% | 100% |
| (%) | | | |

### Experimental Example 2: Phoneme Perception Test for Adult Cochlear Implant Patients

Auditory training methods described in Example 1 were provided to eight adult participants who had undergone cochlear implant surgery, using a smartphone application. This experiment was conducted based on the treatment method of the present invention and followed these steps:
1) Listening to sounds with visual feedback,
2) Training sound recognition by generating sounds through touch and using visual feedback,
3) Training to distinguish incorrect phonemes,
4) Repetitive phoneme recognition training.

Participants conducted rehabilitation by using the application for 30 minutes to 1 hour in a single session. The participants had an average age of 42.88 years (±13.9 years), consisting of one male and seven females. Two participants had congenital hearing loss, while the remaining six had acquired hearing loss. Among them, three were bilateral cochlear implant users, and the remaining five were unilateral cochlear implant users (three left, two right).

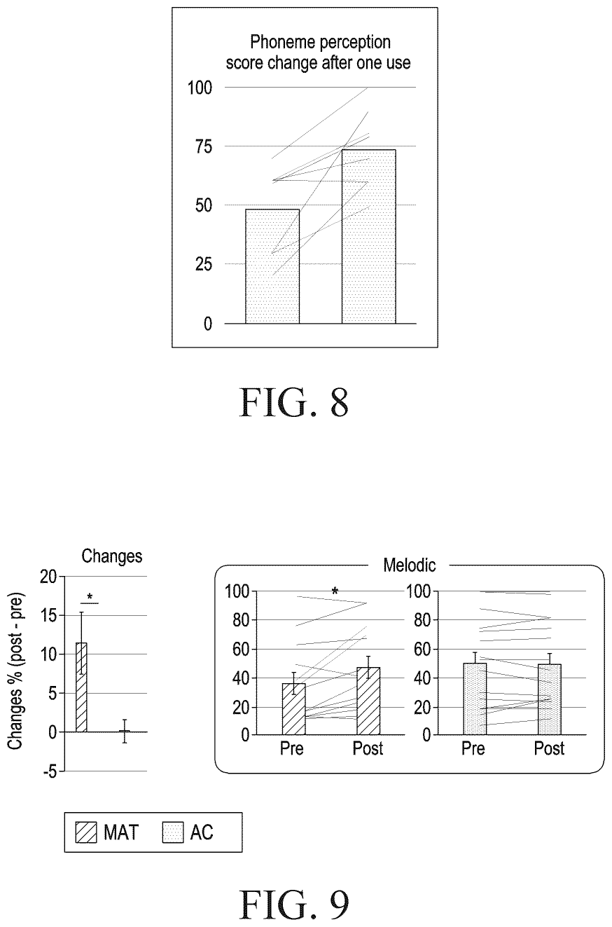

To evaluate the treatment's effectiveness, a speech perception test was conducted before and after treatment, with the results presented in Table 2 and FIG. 8. This test consisted of 10 monosyllabic phonemes, and participants completed the test directly using a smartphone.

As shown in Table 2 and FIG. 8, the average speech perception score before treatment was 48.75 (±18.85), which improved to an average of 73.75 (±16.85) after treatment. To verify the statistical significance of the rehabilitation effects, a paired-sample t-test was conducted. The results confirmed that the changes in scores before and after rehabilitation were statistically significant (t(7)=3.82, p=0.007**).

TABLE 2

| | Pre- | Post- |
|---|---|---|
| | Rehabilitation | Rehabilitation |
| | Score | Score |
| Patient A | 70 | 100 |
| Patient B | 60 | 80 |
| Patient C | 20 | 60 |
| Patient D | 30 | 50 |
| Patient E | 60 | 60 |
| Patient F | 60 | 70 |
| Patient G | 60 | 80 |
| Patient H | 30 | 90 |
| Mean (Standard | 48.75(18.85) | 73.75(16.85) |
| Deviation) | | |

### Experimental Example 3: Multisensory Acoustic Therapy (MAT) for Cochlear Implant Patients

### 3-1. Experimental Design

Thirty-three cochlear implant recipients, aged between 8 and 20 years, who were native Korean speakers, were selected as participants. They were randomly assigned into two groups. The first group (16 participants) underwent Multisensory Acoustic Therapy (MAT), while the second group (17 participants) served as an active control (AC) group and participated in music training.

During the first visit, participants underwent a baseline assessment to evaluate their music and speech perception abilities.

After the baseline visit, participants engaged in their assigned training programs at home for eight weeks. They were instructed to perform the training program for 20-30 minutes per day, five days a week, at their preferred time. Both groups used a smartphone-based application assigned to their respective programs. Participants who completed the training fewer than three times per week on average were excluded from the analysis, leaving a final sample of 28 participants who completed the training and were included in the final analysis.

The MAT group received auditory training through the smartphone application described in Example 2. The AC group was provided with a customized playlist consisting of more than 100 songs across various genres, which they accessed via a commercial music streaming application. To maintain engagement, the playlist was regularly updated.

During the training period, weekly rehabilitation times and application usage data were collected through online surveys to monitor participation.

After the eight-week training period, participants returned to Seoul National University Hospital for a second visit, where follow-up assessments were conducted under the same conditions as the baseline assessment.

### 3-2. Melodic Contour Identification (MCI) Test

To evaluate participants' music perception, the Melodic Contour Identification (MCI) test was used. Participants listened to sequences of five tones presented via a computer and selected the matching pattern from nine possible options. The tone sequences varied in frequency intervals, ranging from 1 to 5 semitones (corresponding to a frequency difference of 5.9% to 33.48%) and were presented in three frequency bands (fundamental frequencies: 220, 440, and 880 Hz).

The test and scoring were conducted using PsychoPy software (version 2014.1.4, Open Science Tools), and the results are presented in Table 1 and FIG. 9. In FIG. 9, the error bars represent the standard error, and the gray lines indicate individual participant changes.

As shown in Table 3 and FIG. 9, the MAT group demonstrated significant improvements in melodic contour identification after training, whereas no significant changes were observed in the AC group.

TABLE 3

| | Time | |
|---|---|---|
| | pre | post |
| MAT | 35.6(27.7) | 47.0(28.7) |
| AC | 48.0(31.6) | 48.1(30.3) |

### 3-3. Speech Perception Test

Participants' speech perception was evaluated through three subtests, each targeting specific linguistic components: monosyllabic word recognition, consonant recognition, and vowel recognition.

The monosyllabic word recognition test consisted of 18 words selected from Korean phonetically balanced word lists, all following a consonant-vowel-consonant (CVC) structure. Responses were scored as correct only when all three components—the initial consonant, vowel, and final consonant—were accurate.

The consonant recognition test used a vowel-consonant-vowel (VCV) format, consisting of 18 items. Scoring focused solely on the accuracy of the consonants provided by participants.

Similarly, the vowel recognition test required participants to identify vowel sounds within a consonant-vowel-consonant (CVC) structure. This test comprised 14 items, with scoring based only on the accuracy of the vowel components.

All speech perception tests utilized standardized recorded stimuli, and the presentation order of the stimuli was randomized. The tests were administered and scored by professional speech therapists who were blinded to the study groups.

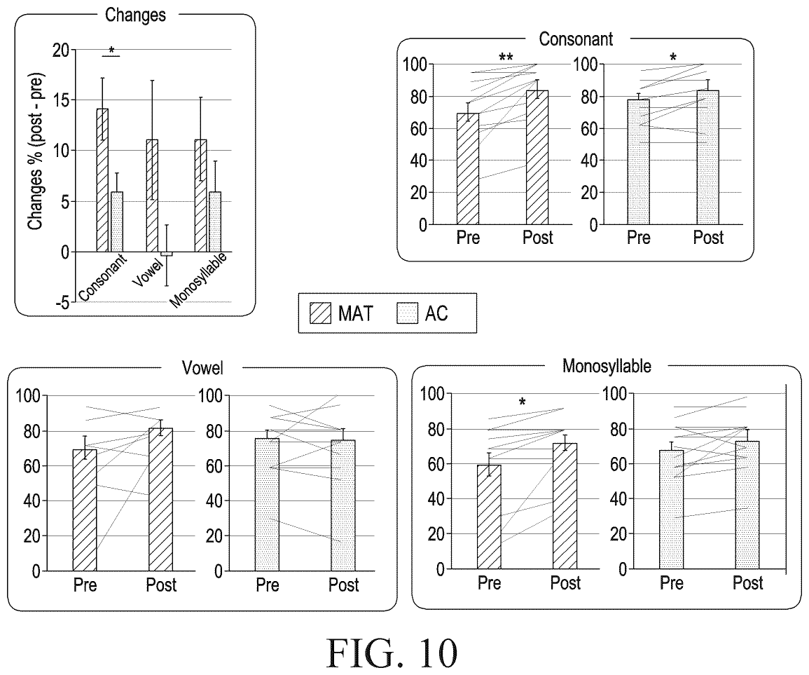

The results are presented in Table 4 and FIG. 10. In FIG. 10, the error bars represent the standard error, and the gray lines indicate individual participant changes.

TABLE 4

| | | Time | |
|---|---|---|---|
| | | pre | post |
| Consonant | MAT | 69.7(19.6) | 83.8(17.3) |
| Identification | AC | 77.0(13.1) | 83.0(15.8) |
| Vowel | MAT | 69.8(25.1) | 80.8(14.7) |
| Identification | AC | 73.3(17.6) | 72.9(21.3) |
| Monosyllable Word | MAT | 58.1(24.8) | 69.2(16.9) |
| Identification | AC | 64.5(16.4) | 70.4(14.7) |

As shown in Table 4 and FIG. 10, both groups demonstrated significant improvements in the consonant recognition test results. However, the MAT group showed greater improvement compared to the AC group. For the vowel recognition and monosyllabic word recognition tests, significant improvements were observed only in the MAT group.

As described above, the hearing loss treatment method according to the present invention enables training that integrates auditory, visual, and motor feedback by providing sound features visually along with sounds for auditory training to hearing-impaired patients and receiving input regarding the sound features from the patient. This method offers a more effective and intuitive learning environment compared to traditional speech-centered training methods, leading to improved auditory enhancement effects. In particular, through multi-sensory auditory training that includes linguistic training, it is possible to comprehensively improve music and language recognition abilities, greatly enhancing hearing-impaired patients' auditory recognition skills and contributing to effective rehabilitation.

Although specific embodiments of the present invention have been described with reference to the attached drawings, those skilled in the art will appreciate that the present invention can be implemented in various specific forms without changing its technical spirit or essential characteristics. Therefore, the embodiments described above should be understood as illustrative and not limiting in any way.

What is claimed is:

1. A method for treating hearing loss through audiovisual interactive auditory training, the method comprising:
(i) providing a test sound having a first auditory pattern and instructing a patient to input a visual pattern corresponding to the test sound;
(ii) providing a first visual object having a first visual pattern to the patient when the patient inputs the first visual pattern, while providing a first sound substantially synchronized with a detection time of the input of the first visual pattern by the patient; and
(iii) providing a second visual object having a second visual pattern corresponding to the first auditory pattern of the test sound.

2. The method of claim 1, further comprising:
(iv) providing a result of a comparison between the first visual object and the second visual object.

3. The method of claim 2,
wherein step (iv) provides information about vulnerable areas of the patient identified based on the result of the comparison.

4. The method of claim 3,
wherein the vulnerable areas of the patient are represented as a value or range of specific frequencies.

5. The method of claim 1,
wherein steps (i) through (iii) are repeated two or more times.

6. The method of claim 5,
wherein when steps (i) through (iii) are repeated, the test sound in a subsequent cycle includes a value or range of frequencies corresponding to vulnerable areas of the patient.

7. The method of claim 1,
wherein the test sound comprises a plurality of segments sequentially provided over time, and each of the plurality of segments changes or remains consistent over time according to the first auditory pattern.

8. The method of claim 1,
wherein the first visual pattern is input by the patient and provided through an interface for inputting the visual pattern.

9. The method of claim 8,
wherein the interface includes a plurality of reference objects for association with the input by the patient.

10. The method of claim 9,
wherein the reference objects are arranged in multiple rows and columns, and
wherein the patient inputs by connecting one of a plurality of first reference objects included in one column to one of a plurality of second reference objects included in an adjacent column.

11. The method of claim 9,
wherein a number, density, and/or arrangement of the reference objects are configured based on a selection by the patient and/or a test difficulty level.

12. The method of claim 1,
wherein the input in step (i) is performed through a touch, drag, and/or click by the patient.

13. The method of claim 1, further comprising:
a training step in which a sound having an auditory pattern is provided, and a visual object having a visual pattern visualizing the auditory pattern of the sound can be listened to and appreciated.

14. The method of claim 1, further comprising a linguistic auditory training step comprising:
(a) providing a test syllable sound composed of one vowel and one consonant, and instructing the patient to input a syllable object corresponding to the test syllable sound;
(b) providing a first syllable sound substantially synchronized with a detection time of the input of a first syllable object by patient; and
(c) providing a second syllable object corresponding to the test syllable sound.

15. The method of claim 14,
wherein step (a) includes a plurality of reference syllable objects for association with the input by the patient.

16. The method of claim 15, further comprising:
prior to step (a), selecting a target phoneme from one vowel and one consonant for training,
wherein the plurality of reference syllable objects include the target phoneme.

17. The method of claim 14,
wherein simultaneously with or after step (c), the method visually provides whether the first syllable object and the second syllable object match.

18. The method of claim 17, further comprising:
a training step in which, if the first syllable object and the second syllable object do not match, the first syllable sound and a second syllable sound can be compared and/or repeatedly listened to.

19. The method of claim 18,
wherein the first syllable sound and the second syllable sound are provided randomly or alternately.

20. The method of claim 19,
wherein the method provides information about vulnerable phonemes of the patient identified based on a result of whether the first syllable object and the second syllable object match.

21. The method of claim 14,
wherein steps (a) through (c) are repeated two or more times.

22. The method of claim 21,
wherein when steps (a) through (c) are repeated, the test syllable sound in a subsequent cycle includes a vulnerable phoneme of the patient.

23. The method of claim 1,
wherein the hearing loss is conductive hearing loss or sensorineural hearing loss.

24. The method of claim 1,
wherein the patient is a user of a cochlear implant.

25. The method of claim 1, further comprising:
administering to the patient at least one selected from the group consisting of steroids, antibiotics, diuretics, and vasodilators.

26. The method of claim 25,
wherein the at least one selected from the group consisting of steroids, antibiotics, diuretics, and vasodilators is administered orally or transdermally.
